(19) United States
(12) Patent Application Publication
(10) Pub. No.: US 2022/0282193 A1
(43) Pub. Date: Sep. 8, 2022

(54) EVALUATION INSTRUMENT AND EVALUATION METHOD

(71) Applicants: THE UNIVERSITY OF TOKYO, Tokyo (JP); TOYOTA BOSHOKU KABUSHIKI KAISHA, Aichi (JP)

(72) Inventors: Shoji TAKEUCHI, Tokyo (JP); Yu SUGIMOTO, Tokyo (JP); Kanae SAKAI, Aichi (JP)

(73) Assignees: THE UNIVERSITY OF TOKYO, Tokyo (JP); TOYOTA BOSHOKU KABUSHIKI KAISHA, Aichi (JP)

(21) Appl. No.: 17/684,915

(22) Filed: Mar. 2, 2022

(30) Foreign Application Priority Data
Mar. 3, 2021 (JP) 2021-033251

(51) Int. Cl. C12M 1/32; C12M 1/00

(52) U.S. Cl. CPC C12M 23/12; C12M 29/18

(57) ABSTRACT

An evaluation instrument is used in evaluating a compound produced by a cell. The evaluation instrument includes a holding space for holding a suspension containing the cell and a through hole that is formed at a bottom of the holding space. The through hole is closed with a substance that is impermeable to the cell and allows the compound to move therethrough.

### CROSS-REFERENCE TO RELATED APPLICATION

This application is based on and claims the priority of Japanese Patent Application No. 2021-033251 filed on Mar. 3, 2021, the entire contents of which are incorporated herein by reference.

### BACKGROUND OF THE INVENTION

### (1) Field of the Invention

The present disclosure relates to an evaluation instrument and an evaluation method.

### (2) Description of Related Art

In order to evaluate a useful compound production capacity of a cell, for example, a microchamber made of PDMS is used (see JP 2020-202777 A). Specifically, first, cells are cultured in a plurality of wells using a plate-shaped microchamber having the wells on its surface. Then, the microchamber is placed, in a reversed state, on a medium coated with a test bacterium to transfer an antibacterial component and the like produced by the cells to the medium. Next, a degree of suppression of growth of the test bacterium in the medium to which the antibacterial component and the like have been transferred is observed to evaluate, for example, an antibacterial substance production capacity of the cells.

### SUMMARY OF THE INVENTION

However, there is a possibility that this method may provide low accuracy of evaluation, because the cells that produce the antibacterial component and the like are also transferred together when the microchamber is placed, in a reversed state, on the medium coated with the test bacterium.

The present disclosure has been made in view of the above circumstances, and an object thereof is to improve the accuracy of evaluation. The present disclosure can be realized as the following forms.

[1] An evaluation instrument for a compound produced by a cell, including:

a holding space for holding a suspension containing the cell, and

a through hole that is formed at a bottom of the holding space,

wherein the through hole is closed with a substance that is impermeable to the cell and allows the compound to move therethrough.

According to the evaluation instrument of the present disclosure, the substance disposed in the through hole allows the compound produced by the cell to move to the medium and does not allow the cell itself to move to the medium, so that the accuracy of evaluation can be improved.

### BRIEF DESCRIPTION OF THE DRAWINGS

FIG. 1 is a perspective view illustrating an example of an evaluation instrument;

FIG. 2 is a plan view illustrating the example of the evaluation instrument;

FIG. 3 is a partially enlarged view of FIG. 1;

FIG. 4 is a cross-sectional view illustrating the example of the evaluation instrument;

FIG. 5 is a perspective view illustrating an example of the evaluation instrument with a lid;

FIG. 6 is a schematic diagram illustrating the evaluation instrument into which a suspension is injected from an introduction port;

FIG. 7 is a schematic diagram illustrating the evaluation instrument into which air is injected from the introduction port;

FIG. 8 is a schematic diagram (cross-sectional view) illustrating an example of an evaluation method;

FIG. 9 is a schematic diagram (cross-sectional view) illustrating an example of the evaluation method;

FIG. 10 is a schematic diagram (cross-sectional view) illustrating an example of the evaluation method;

FIG. 11 is a schematic diagram (cross-sectional view) illustrating an example of the evaluation method; and

FIG. 12 is a perspective view illustrating an example of an evaluation instrument according to another embodiment.

### DETAILED DESCRIPTION OF PREFERRED EMBODIMENTS

Here, desirable examples of the present disclosure are presented.

[2] The evaluation instrument, wherein a plurality of the holding spaces is present.

A number n can be increased to improve the evaluation accuracy.

[3] The evaluation instrument, wherein the through hole is formed independently for each of the holding spaces.

Independency of each compound produced in each holding space is ensured.

[4] The evaluation instrument, wherein the holding spaces are connected to each other.

The plurality of holding spaces can be easily filled with the suspension.

[5] The evaluation instrument, wherein an introduction portion for introducing the suspension into the holding space is formed, and the holding space is connected to a flow path extending from the introduction portion.

The plurality of holding spaces can be easily filled with the suspension.

[6] The evaluation instrument, wherein the flow path extends linearly, and the holding spaces are connected to either side of the flow path.

The plurality of holding spaces can be substantially uniformly filled with the suspension.

[7] An evaluation method including:

placing the evaluation instrument according to claim 1 on a medium to bring the substance closing the through hole into contact with the medium; and

allowing the compound produced by the cell to move to the medium via the substance to evaluate the compound in the medium.

The substance disposed in the through hole allows the compound produced by the cell to move to the medium, and does not allow the cell itself to move to the medium, so that the accuracy of the evaluation method is improved.

[8] An evaluation method including:

placing the evaluation instrument according to claim 1 on a medium coated with a bacterium to bring the substance closing the through hole into contact with the medium; and

allowing the compound produced by the cell to move to the medium via the substance to observe states of a cloudy region and a transparent region formed on the medium, thereby evaluating the compound.

The substance disposed in the through hole allows the compound produced by the cell to move to the medium, and does not allow the cell itself to move to the medium, so that the accuracy of evaluation is improved.

Hereinafter, the present disclosure will be described in detail. In the present specification, a phrase about a numerical range using the word “to” includes a lower limit value and an upper limit value unless otherwise specified. For example, the phrase “10 to 20” includes both the lower limit “10” and the upper limit “20”. That is, the phrase “10 to 20” has the same meaning as “10 or more and 20 or less”.

### 1. Evaluation Instrument 1

An evaluation instrument 1 of the present disclosure is used in evaluating a compound 3 produced by a cell.

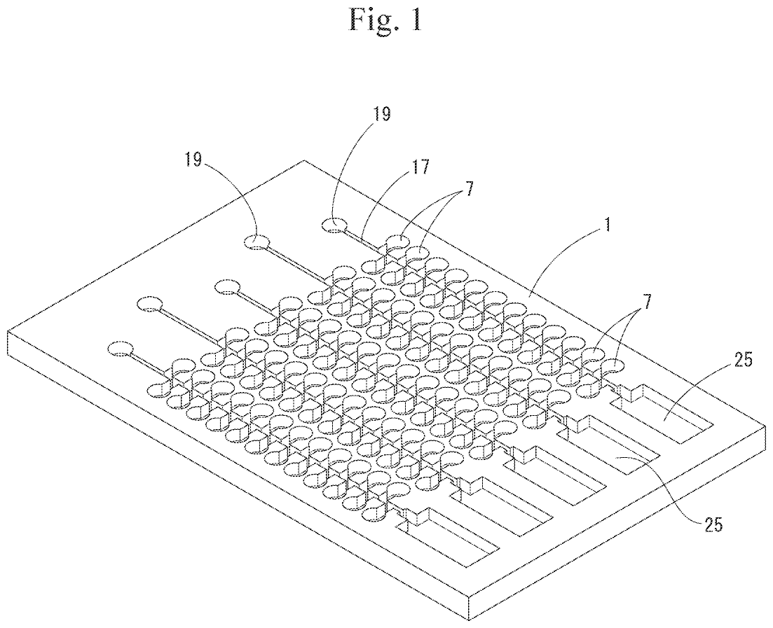

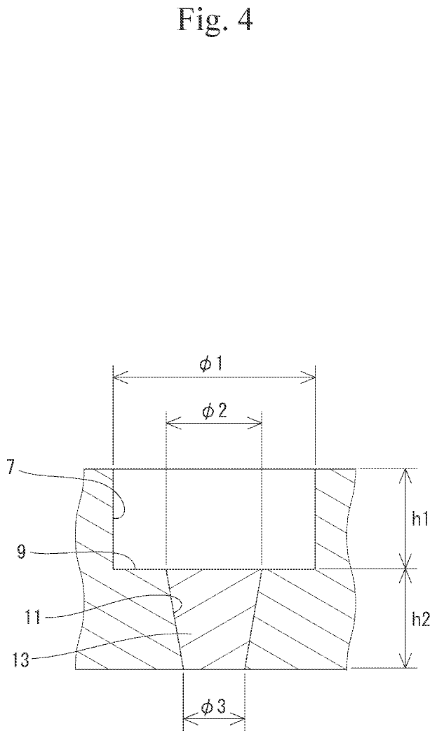

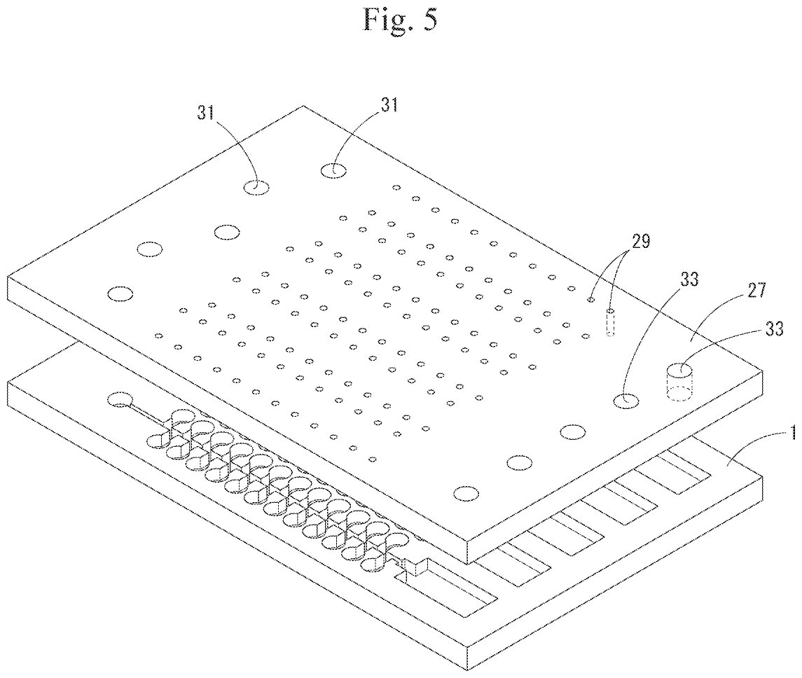

The evaluation instrument 1 is provided with a holding space 7 for holding a suspension 5 containing the cell, as illustrated in FIGS. 1 to 4. A through hole 11 is formed at a bottom 9 of the holding space 7. The through hole 11 is closed with a substance 13 that is impermeable to the cell and allows the compound 3 to move therethrough. FIGS. 1, 2, 3, and 5 illustrate a state before the through hole 11 is closed with the substance 13, and FIG. 4 illustrates a state in which the through hole 11 is closed with the substance 13. The evaluation instrument 1 in use is in a state in which the through hole 11 is closed with the substance 13.

A number of the holding spaces 7 is not particularly limited. From the viewpoint of increasing the number n to improve the evaluation accuracy, it is preferable that a plurality of the holding spaces 7 should be present.

A shape of the holding space 7 is not particularly limited. The holding space 7 preferably has a substantially columnar shape, from the viewpoint of smoothly filling the holding space 7 with the suspension 5. When the holding space 7 has a substantially columnar shape, a diameter φ1 and a height h1 of the holding spaces 7 are not particularly limited. From the viewpoint of allowing a cell to produce a sufficient amount of the compound 3 for evaluation, the diameter φ1 is preferably 0.8 mm to 1.2 mm, and the height h1 is preferably 0.3 mm to 0.7 mm.

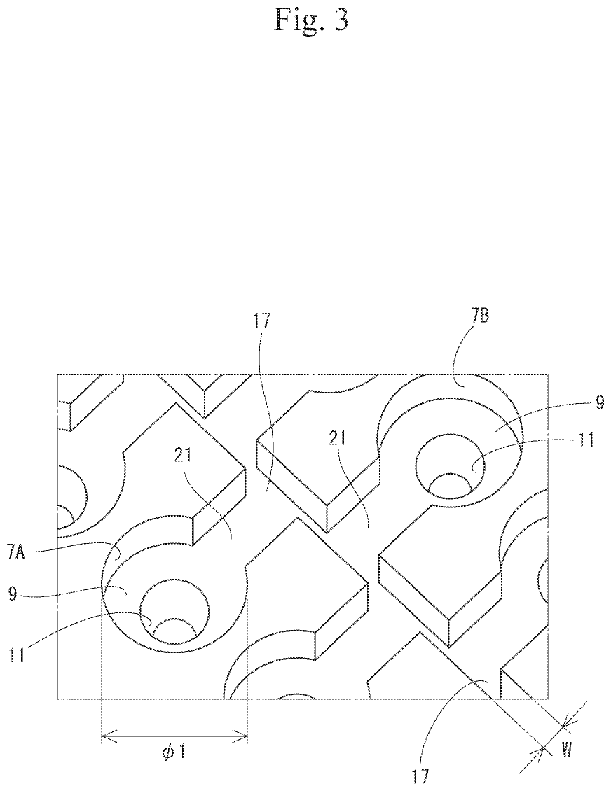

As illustrated in FIG. 3, the through hole 11 is preferably formed independently for each holding space 7. This is intended to ensure independency of each compound 3 produced in each holding space. A shape of the through hole 11 is not particularly limited. As illustrated in FIG. 4, the through hole 11 preferably has a shape in which a diameter decreases downward, for example, a truncated cone shape. With this shape, the substance 13 is prevented from falling off from the through hole 11. When the through hole 11 has a truncated cone shape, a diameter φ2 of its upper end portion is preferably 0.25 mm to 0.35 mm, a diameter φ3 of its lower end portion is preferably smaller than the diameter φ2, and a height h2 is preferably 0.3 mm to 0.7 mm, from the viewpoint of sufficiently securing a movement amount of the compound 3 in the substance 13 while suppressing falling off of the substance 13.

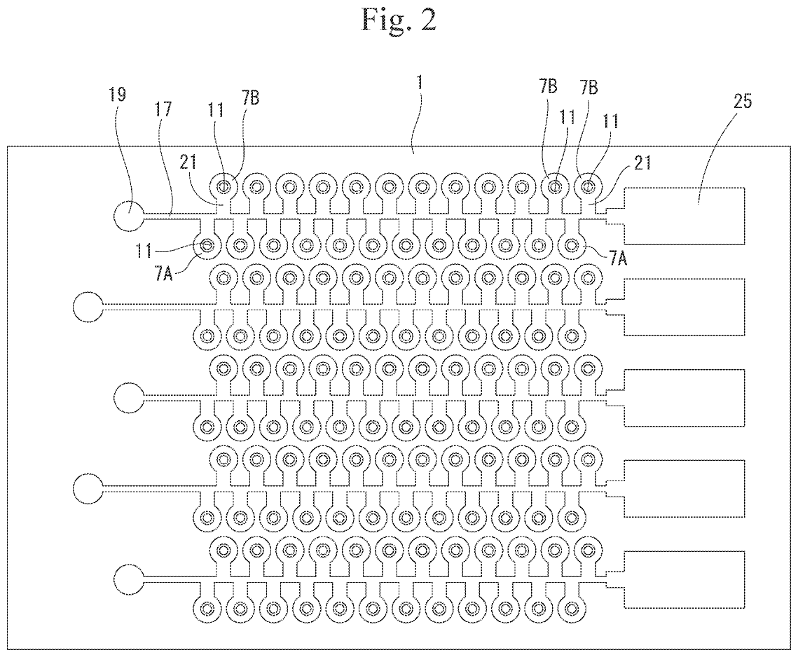

When a plurality of the holding spaces 7 is present, holding spaces 7 are preferably connected to each other, as illustrated in FIGS. 2 and 3. In the examples illustrated in FIGS. 2 and 3, the plurality of holding spaces 7 are connected by a flow path 17 and connection flow paths 21 so that liquid can flow therethrough.

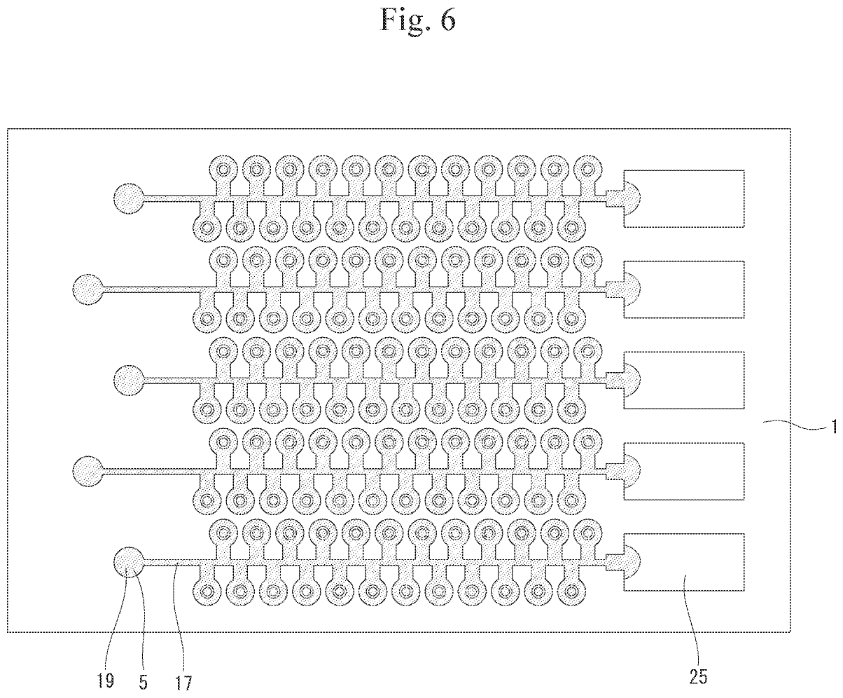

In the evaluation instrument 1, an introduction portion 19 for introducing the suspension 5 into the holding spaces 7 is formed. The holding spaces 7 are connected to the flow path 17 extending from the introduction portion 19 via the connection flow paths 21. In this configuration, the suspension 5 can be easily filled in the plurality of holding spaces 7 by injecting the suspension 5 into the introduction portion 19 (see FIG. 6).

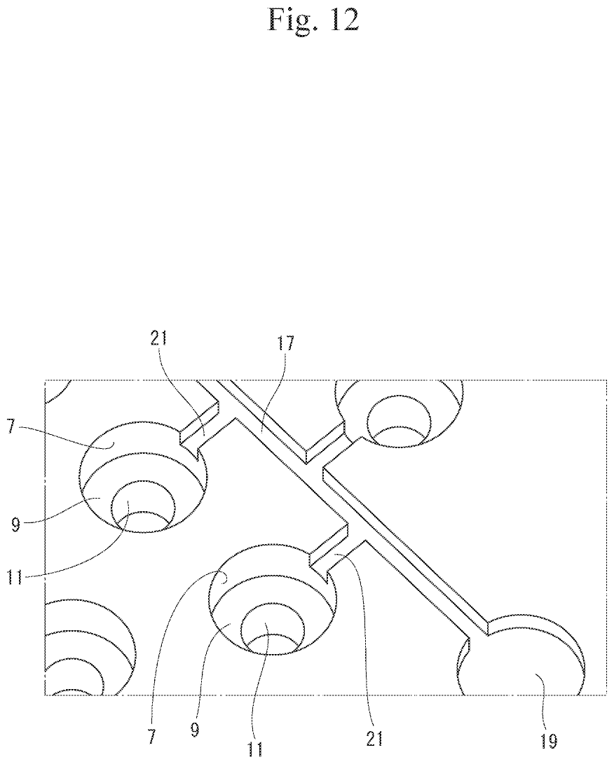

The flow path 17 extends linearly (for example, straight), and the holding spaces 7 are preferably connected to either side of the flow path 17. The flow path 17 and the holding spaces 7 are connected by the connection flow paths 21. In FIG. 3, the flow path 17 and the connection flow path 21 have the same height as the bottom 9 of the holding space 7, but, as illustrated in FIG. 12, the flow path 17 and the connection flow path 21 may have different heights from the bottom 9 of the holding space 7. In FIG. 12, the bottom 9 of the holding space 7 is positioned lower than the flow path 17 and the connection flow path 21.

The arrangement of the plurality of holding spaces 7 on either side of the flow path 17 is not particularly limited. As illustrated in FIG. 2, holding spaces 7A located on one side of the flow path 17 and holding spaces 7B located on the other side of the flow path 17 are alternately arranged along a direction from the introduction portion 19 to a discharge portion 25. With this structure, when the suspension 5 is introduced from the introduction portion 19, the holding spaces 7A located on one side of the flow path 17 and the holding spaces 7B located on the other side of the flow path 17 are alternately filled with the suspension 5, so that the suspension 5 can be substantially uniformly poured into each holding space 7.

A width W of the flow path 17 is not particularly limited. The width W of the flow path 17 is set to, for example, 0.1 mm or more and 0.3 mm or less, from the viewpoint of smoothly flowing the suspension 5 into the flow path 17.

The evaluation instrument 1 is preferably provided with a lid 27. In the lid 27, a vent hole 29 is formed at a position above each holding space 7. In the lid 27, an introduction port 31 is formed at a position above the introduction portion 19, and a discharge port 33 is formed at a position above the discharge portion 25.

The material of the evaluation instrument 1 is not particularly limited. As the material, for example, a material containing a silicone resin as a main component is suitably used. Here, the main component refers to a substance having a content rate (mass %) of 50 mass % or more. The silicone resin is not particularly limited, but is preferably polydimethylsiloxane (PDMS), polymethylphenylsiloxane, polymethylhydrogensiloxane, polymethylmethoxysiloxane, polymethylvinylsiloxane, and the like. These silicone resins may be used singly, or two or more thereof may be used in combination. Preference is given to polydimethylsiloxane (PDMS). The lid 27 can also be suitably made of the same material as that of the evaluation instrument 1.

The compound 3 produced by the cell is not particularly limited. The compound 3 is, for example, a useful substance such as an antibacterial substance (antibiotic). The cell that produces the compound 3 is also referred to as producing cell.

The substance 13 is impermeable to the cell and allows the compound 3 to move therethrough. As the substance 13, agar (agar gel) can be suitably employed.

According to the evaluation instrument 1 of the present disclosure, the substance 13 disposed in the through hole 11 allows the compound 3 produced by the cell to move to the medium 35, and does not allow the cell itself to move to the medium 35, so that the accuracy of evaluation is improved. That is, the evaluation instrument 1 of the present disclosure can be expected to provide the following effect. According to the evaluation instrument 1, since the producing cell does not move to the medium 35, there is little possibility that the producing cell and a detection bacterium (test bacterium) may be mixed in the medium 35. Therefore, the evaluation instrument 1 makes it possible to appropriately evaluate growth failure or the like of the detection bacterium (test bacterium) in the medium 35.

### 2. Evaluation Method

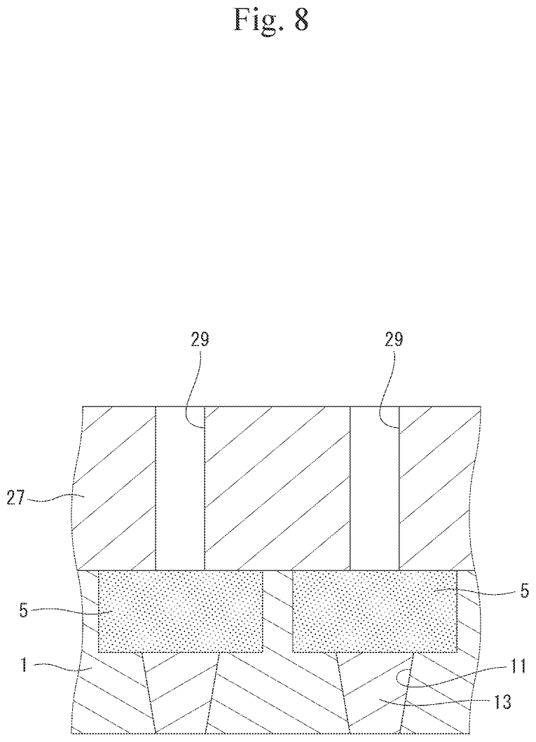

An evaluation method of the present disclosure includes: placing the evaluation instrument 1 on the medium 35 to bring the substance 13 closing the through hole 11 into contact with the medium 35; and allowing the compound 3 produced by the cell to move to the medium 35 via the substance 13 to evaluate the compound 3 in the medium 35 (see FIGS. 8 to 11).

As the medium 35, for example, an agar medium is suitably employed. From the viewpoint of handleability, the agar medium is preferably a solid medium, and more preferably a flat plate medium. The agar medium is preferably coated with a bacterium. The bacterium is also called detection bacterium (test bacterium), and a pathogenic bacterium or the like is suitably employed.

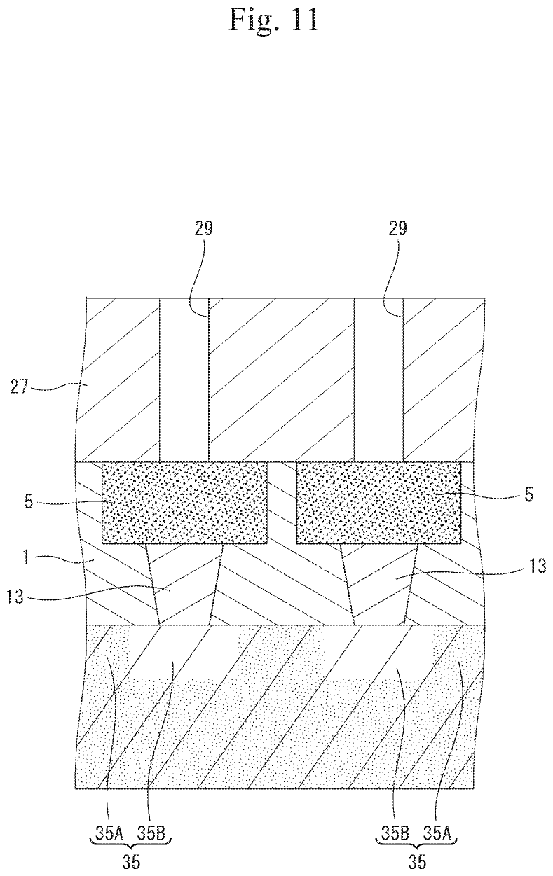

An evaluation technique is not particularly limited. As the evaluation technique, for example, as illustrated in FIG. 11, the compound 3 is evaluated by observing states of a cloudy region 35A and a transparent region 35B formed on the medium 35.

According to the evaluation method of the present disclosure, the substance 13 disposed in the through hole 11 allows the compound 3 produced by the cell to move to the medium 35, and the cell itself does not move to the medium 35, so that the accuracy of evaluation is improved. That is, the evaluation method of the present disclosure can be expected to provide the following effect. According to the evaluation method, since the producing cell does not move to the medium 35, there is little possibility that the producing cell and a detection bacterium (test bacterium) may be mixed in the medium 35. Therefore, the evaluation method makes it possible to appropriately evaluate growth failure or the like of the detection bacterium (test bacterium) in the medium 35.

Here, a preferable evaluation method will be described in more detail.

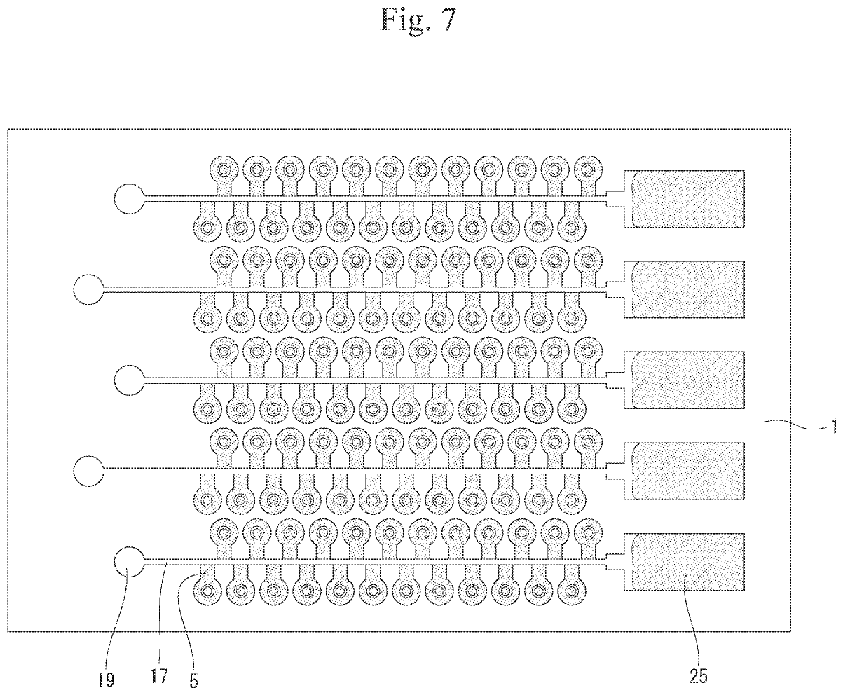

The evaluation instrument 1 whose upper surface is closed with the lid 27 is used. In FIGS. 6 and 7, the lid 27 is omitted for easy understanding of the description. When the suspension 5 is injected from the introduction port 31, the suspension 5 which is schematically illustrated by stippling is introduced from the introduction portion 19, as illustrated in FIG. 6. The suspension 5 smoothly flows through the linear flow path 17. The holding spaces 7 are filled with the suspension 5 in the ascending order of the distance from the introduction portion 19.

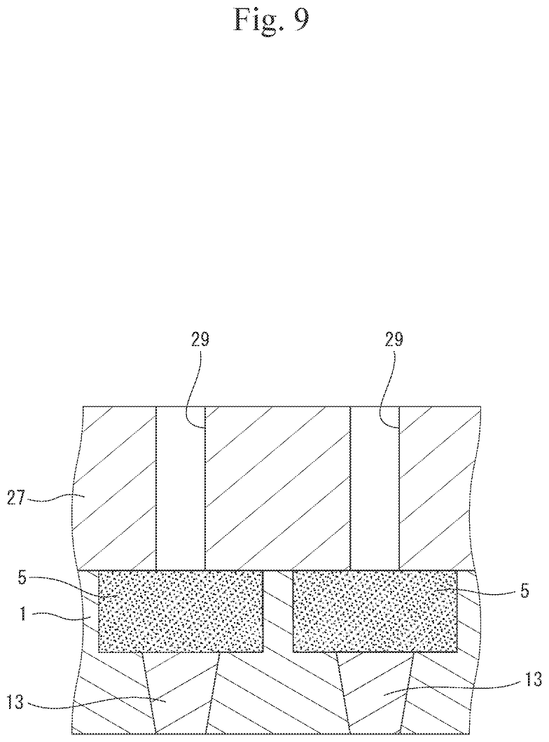

Next, when air is injected from the introduction port 31, the air is injected from the introduction portion 19 as illustrated in FIG. 7. Then, the air travels substantially straight, the suspension 5 that has remained in the linear flow path 17 is smoothly pushed out to the discharge portion 25, so that the suspension 5 filled in each holding space 7 becomes an unconnected state. That is, the suspensions 5 filled in the respective holding spaces 7 become independent of each other. FIG. 8 is a cross-sectional view of this state. Then, when the cells in the suspension 5 are cultured in this state, the compound 3 is generated in the suspension 5 as illustrated in FIG. 9. A density of dots in stippling of the suspension 5 in FIG. 9 is higher than a density of dots in stippling of the suspension 5 in FIG. 8, which schematically indicates that the compound 3 has been generated in the suspension 5 in FIG. 9.

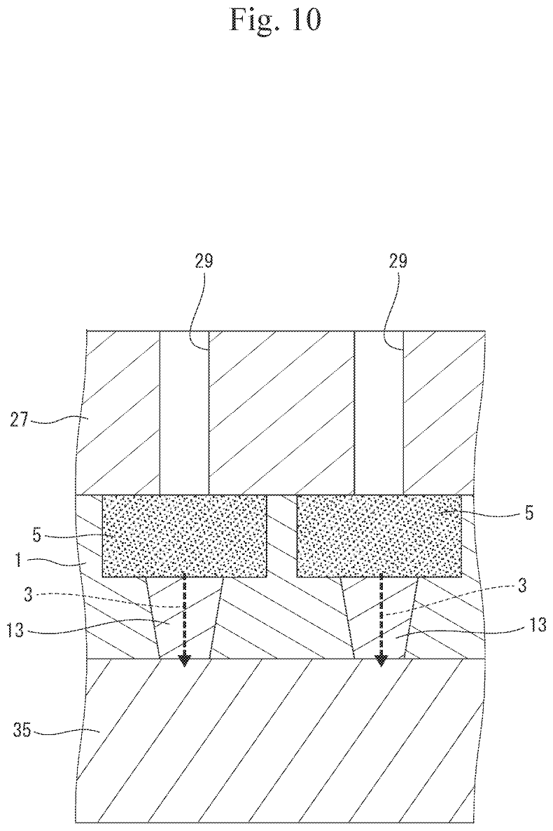

Separately, the medium 35 coated with a bacterium (not illustrated) is prepared. As illustrated in FIG. 10, the evaluation instrument 1 is placed on the medium 35 to bring a lower surface of the substance 13 and an upper surface of the medium 35 into contact with each other. The compound 3 produced by the cells moves (diffuses) into the medium 35 via the substance 13 as indicated by dashed arrows in FIG. 10.

Thereafter, the medium 35 is kept at a temperature suitable for culturing (proliferating) the detection bacterium coated onto the medium 35, for example, 20° C. to 45° C., and more preferably 35° C. to 40° C.

Then, the medium 35 is observed. For example, the states of the cloudy region 35A and the transparent region 35B (halo) formed on the medium 35 are observed.

When the compound 3 is an antibiotic effective against the detection bacterium, in the medium 35, a periphery of a region in contact with the substance 13 becomes the transparent region 35B, and the other region becomes the cloudy region 35A (opaque region). In this case, the presence of the transparent region 35B means that the growth of the bacterium (detection bacterium) is inhibited by the antibiotic. That is, the observation of these transparent region 35B and cloudy region 35A makes it possible to confirm that the bacterium is killed by the presence of the antibiotic and that the bacterium is proliferated by the absence of the antibiotic, and, eventually, the antibacterial activity of the antibiotic produced by the cells can be evaluated.

It is noted that the foregoing examples have been provided merely for the purpose of explanation and are in no way to be construed as limiting of the present disclosure. While the present disclosure has been described with reference to exemplary embodiments, it is understood that the words which have been used herein are words of description and illustration, rather than words of limitation. Changes may be made, within the scope of the appended claims, without departing from the scope and spirit of the present disclosure in its aspects. Although the present disclosure has been described herein with reference to particular structures, materials and embodiments, the present disclosure is not intended to be limited to the particulars disclosed herein; rather, the present disclosure extends to all functionally equivalent structures, methods and uses, which are within the scope of the appended claims.

The present disclosure is not limited to the embodiments described in detail above, and can be modified or changed in various manners within the scope as set forth in the claims of the present disclosure.

The evaluation instrument of the present disclosure can be used in evaluating a compound produced by a cell.

What is claimed is:

1. An evaluation instrument for a compound produced by a cell, comprising:
a holding space for holding a suspension containing the cell, and
a through hole that is formed at a bottom of the holding space,
wherein the through hole is closed with a substance that is impermeable to the cell and allows the compound to move therethrough.

2. The evaluation instrument according to claim 1, wherein a plurality of the holding spaces is present.

3. The evaluation instrument according to claim 2, wherein the through hole is formed independently for each of the holding spaces.

4. The evaluation instrument according to claim 2, wherein the holding spaces are connected to each other.

5. The evaluation instrument according to claim 1, wherein an introduction portion for introducing the suspension into the holding space is formed, and the holding space is connected to a flow path extending from the introduction portion.

6. The evaluation instrument according to claim 5, wherein the flow path extends linearly, and the holding spaces are connected to either side of the flow path.

7. An evaluation method comprising:
placing the evaluation instrument according to claim 1 on a medium to bring the substance closing the through hole into contact with the medium; and
allowing the compound produced by the cell to move to the medium via the substance to evaluate the compound in the medium.

8. An evaluation method comprising:
placing the evaluation instrument according to claim 1 on a medium coated with a bacterium to bring the substance closing the through hole into contact with the medium; and
allowing the compound produced by the cell to move to the medium via the substance to observe states of a cloudy region and a transparent region formed on the medium, thereby evaluating the compound.
